(19) United States
(12) Patent Application Publication
(10) Pub. No.: US 2025/0076845 A1
(43) Pub. Date: Mar. 6, 2025

(54) SERVER, METHOD, PROGRAM AND COMPUTER-READABLE RECORDING MEDIUM FOR OPERATING 3-DIMENSIONAL DENTAL LAMINATE MODIFICATION FOR ATTACHING ORNAMENTS

(71) Applicant: e32 Technology Inc., Chantilly, VA (US)

(72) Inventors: Young Cheol CHANG, Fairfax, VA (US); Junhyuk YOON, Seoul (KR); Hyunjin LEE, Chantilly, VA (US)

(73) Assignee: e32 Technology Inc., Chantilly, VA (US)

(21) Appl. No.: 18/240,916

(22) Filed: Aug. 31, 2023

(51) Int. Cl. G05B 19/4099; G06T 7/50; G06T 7/64; G06T 19/20

(52) U.S. Cl. CPC G05B 19/4099; G06T 7/50; G06T 7/64; G06T 19/20; G05B 2219/49023; G06T 2207/30196; G06T 2219/2004; G06T 2219/2021

(57) ABSTRACT

Disclosed are a server, a method, and a computer-readable recording medium for operating a three-dimensional dental laminate modification platform for adhering ornaments thereto. The server includes a processor and a memory operably connected to the processor, wherein instructions stored in the memory, when executed by the processor, cause the processor to: receive a three-dimensional dental laminate modeling file; receive an ornament modeling file; determine an adhered surface of the ornament to be adhered to the three-dimensional laminate in the received ornament modeling file, and extract an image of the adhered surface therefrom; and upon determination that the three-dimensional dental laminate modeling file is required to be modified, form an intaglio based on the extracted image of the adhered surface in a position on the laminate in which the ornament is to be adhered thereto in the three-dimensional dental laminate modeling file, thereby modifying the three-dimensional dental laminate modeling file.

### BACKGROUND

### Field

The present disclosure relates to a server, a method, a program, and a computer-readable recording medium for operating a three-dimensional dental laminate modification platform for adhering ornaments thereto using image information.

### Description of Related Art

Unless otherwise indicated herein, materials as described in this Background section are not prior art to the claims of the present application. Despite the fact that the materials are included in this Background section, the materials are not admitted to be prior art.

The esthetics and functions of teeth continue to play an important role in oral health care and cosmetic purposes. In the past, invasive treatment to improve tooth function, such as dental treatment such as implants, has been frequently performed. Although the invasive treatment is effective, it causes unpredictable risks or involves an irreversible dental procedure that may limit a future possible treatment option.

With the recent development and opening of culture, research has been conducted to achieve aesthetically advanced dental treatment outcomes via non-invasive and simple procedures to improve the appearance of teeth. In particular, 3D scanning and 3D printing technology of teeth has been proposed as a scheme of increasing the effectiveness of non-invasive dental treatment. Further, a system for fabricating a three-dimensional dental laminate that may be used for cosmetic purposes in daily life by creating a dental laminate with a level of precision similar to that of the prior art only using image information, such as photos and/or videos of the user's teeth imaged via a mobile phone, etc. without visiting the dentist is being researched and developed.

Furthermore, tooth gems such as ornaments made of various materials such as cubic, metal, or plastic, and adhered to teeth using dental adhesives for aesthetic purposes or user's preference, are in vogue. However, when a flat adhered surface of the ornament with no curvature as a rear surface thereof is adhered to an adhered surface of a tooth with a curvature, or when the curvature of the adhered surface of the ornament does not match the curvature of the adhered surface of the tooth, the adhesive strength may be degraded and the damage to the tooth may occur during the bonding process. Further, there are disadvantages in that the user cannot easily attach or detach the ornament to or from the tooth depending on the situation or place, and that the user's health may be adversely affected when the ornament is separated from the teeth due to the decrease in the adhesive strength.

Accordingly, a study on a three-dimensional dental laminate that the user can attach or detach easily the ornament to or from, and may increase the aesthetics of the user's teeth via solid adhesion of the ornament thereto without damaging the user's real teeth is in need.
Prior art literature may be Patent Literature: Korean Patent No. 10-2244439

### SUMMARY

A purpose of the present disclosure is to provide a server, a method, a program, and a computer-readable recording medium for operating a three-dimensional dental laminate modification platform for adhering various types of ornaments to the 3D dental laminate registered on a real tooth as generated using image information.

A first aspect of the present disclosure to achieve the above purpose provides a server for operating a three-dimensional dental laminate modification platform for adhering ornaments thereto.

The server for operating a three-dimensional dental laminate modification platform for adhering an ornament to a laminate comprises: at least one processor, and a memory operably connected to the at least one processor, wherein the memory stores therein instructions, wherein when the instructions are executed by the at least one processor, the instructions cause the at least one processor to: receive a three-dimensional dental laminate modeling file; receive an ornament modeling file; determine an adhered surface of the ornament to be adhered to the three-dimensional laminate in the received ornament modeling file, and extract an image of the adhered surface therefrom; and upon determination that the three-dimensional dental laminate modeling file is required to be modified, form an intaglio based on the extracted image of the adhered surface in a position on the laminate in which the ornament is to be adhered thereto in the three-dimensional dental laminate modeling file, thereby modifying the three-dimensional dental laminate modeling file.

In one implementation of the first aspect, when the instructions are executed by the at least one processor, the instructions further cause the at least one processor to: extract, as height information, a thickness of the ornament in a normal direction to the adhered surface thereof; and determine a depth of the formed intaglio as a predetermined percentage of the height information.

In one implementation of the first aspect, when the instructions are executed by the at least one processor, the instructions further cause the at least one processor to: identify a plurality of teeth included in the received three-dimensional dental laminate modeling file; calculate a curvature of an outer surface of each of the identified plurality of teeth; and determine a tooth having the calculated curvature smaller than a predetermined curvature as a tooth to which the ornament can be adhered.

In one implementation of the first aspect, when the instructions are executed by the at least one processor, the instructions further cause the at least one processor to modify the ornament modeling file such that a curvature of the adhered surface of the ornament is equal to a curvature of a tooth at a position on the three-dimensional dental laminate at which the ornament is adhered to the laminate.

In one implementation of the first aspect, when the instructions are executed by the at least one processor, the instructions further cause the at least one processor to receive a position on the three-dimensional dental laminate at which the ornament is to be adhered to the laminate.

In one implementation of the first aspect, when the instructions are executed by the at least one processor, the instructions further cause the at least one processor to: receive three-dimensional dental laminate information; classify the received three-dimensional dental laminate information into at least one category based on attributes thereof; combine the three-dimensional dental laminate information included in each classified category with each other to calculate a dental laminate score; and determine a recommended ornament grade based on the calculated dental laminate score and recommend the ornament based on the grade.

In one implementation of the first aspect, when the instructions are executed by the at least one processor, the instructions further cause the at least one processor to: receive ornament information; classify the received ornament information into at least one category based on attributes thereof; combine the ornament information included in each classified category with each other to calculate an ornament physical property score; and classify the ornament into an ornament grade set to a predetermined score range, based on the calculated ornament physical property score.

In one implementation of the first aspect, when the instructions are executed by the at least one processor, the instructions further cause the at least one processor to recommend an ornament having the ornament grade classified based on the calculated ornament physical property score and the recommended ornament grade classified based on the dental laminate score equal to each other as an ornament suitable for the three-dimensional dental laminate.

In one implementation of the first aspect, the calculating of the ornament physical property score includes: dividing a multiplication of an adhesion size, flexibility and hardness of the ornament by a multiplication of an adhesion weight, thermal conductivity, and thermal expansion coefficient of the ornament to obtain the ornament physical property score; and performing minimum-maximum normalization based on a predetermined minimum ornament physical property score and a predetermined maximum ornament physical property score to obtain a normalized ornament physical property score.

A second aspect of the present disclosure to achieve the above purpose provides a method for modifying a three-dimensional dental laminate for adhering an ornament to the laminate, wherein the method is carried out in a server for operating a three-dimensional dental laminate modification platform for adhering the ornament to the laminate.

The method for modifying a three-dimensional dental laminate for adhering an ornament to the laminate comprises: receiving a three-dimensional dental laminate modeling file; receiving an ornament modeling file; determining an adhered surface of the ornament to be adhered to the three-dimensional laminate in the received ornament modeling file, and extracting an image of the adhered surface therefrom; and upon determination that the three-dimensional dental laminate modeling file is required to be modified, forming an intaglio based on the extracted image of the adhered surface in a position on the laminate in which the ornament is to be adhered thereto in the three-dimensional dental laminate modeling file, thereby modifying the three-dimensional dental laminate modeling file.

In one implementation of the second aspect, the method further comprises: extracting, as height information, a thickness of the ornament in a normal direction to the adhered surface thereof; and determining a depth of the formed intaglio as a predetermined percentage of the height information.

In one implementation of the second aspect, the method further comprises: identifying a plurality of teeth included in the received three-dimensional dental laminate modeling file; calculating a curvature of an outer surface of each of the identified plurality of teeth; and determining a tooth having the calculated curvature smaller than a predetermined curvature as a tooth to which the ornament can be adhered.

In one implementation of the second aspect, the method further comprises modifying the ornament modeling file such that a curvature of the adhered surface of the ornament is equal to a curvature of a tooth at a position on the three-dimensional dental laminate at which the ornament is to be adhered to the laminate.

In one implementation of the second aspect, the method further comprises receiving a position on the three-dimensional dental laminate at which the ornament is to be adhered to the laminate.

In one implementation of the second aspect, the method further comprises: receiving three-dimensional dental laminate information; classifying the received three-dimensional dental laminate information into at least one category based on attributes thereof; combining the three-dimensional dental laminate information included in each classified category with each other to calculate a dental laminate score; and determining a recommended ornament grade based on the calculated dental laminate score and recommending the ornament based on the grade.

In one implementation of the second aspect, the method further comprises: receiving ornament information; classifying the received ornament information into at least one category based on attributes thereof; combining the ornament information included in each classified category with each other to calculate an ornament physical property score; and classifying the ornament into an ornament grade set to a predetermined score range, based on the calculated ornament physical property score.

In one implementation of the second aspect, the method further comprises recommending an ornament having the ornament grade classified based on the calculated ornament physical property score and the recommended ornament grade classified based on the dental laminate score equal to each other as an ornament suitable for the three-dimensional dental laminate.

In one implementation of the second aspect, the calculating of the ornament physical property score includes: dividing a multiplication of an adhesion size, flexibility and hardness of the ornament by a multiplication of an adhesion weight, thermal conductivity, and thermal expansion coefficient of the ornament to obtain the ornament physical property score; and performing minimum-maximum normalization based on a predetermined minimum ornament physical property score and a predetermined maximum ornament physical property score to obtain a normalized ornament physical property score.

In one implementation of the second aspect, the method further comprises transmitting the modified three-dimensional dental laminate modeling file to a 3D printer connected to the platform over a wired/wireless network such that the 3D printer fabricates a modified three-dimensional dental laminate.

A third aspect of the present disclosure to achieve the above purpose provides a computer-readable recording medium storing therein a computer program, wherein when the computer program is executed by a server including at least one processor, the computer program causes the at least one processor to perform the method for modifying the three-dimensional dental laminate for adhering the ornament thereto as described above.

### Effects of the Invention

According to an embodiment of the present disclosure, the ornament that can be adhered to the 3D dental laminate may be adhered to the tooth without irreversible loss of the tooth. The ornament may be adhered to the adhered surface of the 3D dental laminate in a registered manner thereon such that the ornament can be firmly adhered thereto, and oral health of the user may be maintained. The effects obtainable from the present disclosure are not limited to the effects mentioned above. Rather, other effects not mentioned will be clearly understood by those skilled in the art from descriptions below.

### BRIEF DESCRIPTION OF DRAWINGS

Other aspects, features and benefits other than those as described above of certain preferred embodiments of the present disclosure will become more apparent from the following descriptions taken together with the accompanying drawings.

FIG. 1 is an illustrative view showing an operating environment of a server for operating a three-dimensional dental laminate modification platform for adhering ornaments thereto according to an embodiment of the present disclosure.

FIG. 2 is an illustrative diagram showing an example of a server for operating a three-dimensional dental laminate modification platform for adhering ornaments thereto.

FIG. 3 is an illustrative view to illustrate an example of customizing a three-dimensional dental laminate modeling file based on a shape of an ornament.

FIG. 4 is a detailed illustrative diagram for illustrating customization of a modeling file according to an embodiment of the present disclosure.

FIG. 5 is an illustrative diagram showing another example of a server for operating a three-dimensional dental laminate modification platform for adhering ornaments thereto.

FIG. 6 is an illustrative view to illustrate an example of manufacturing a three-dimensional dental laminate and ornament.

FIG. 7 is an illustrative flow chart to illustrate a method in which a server for operating a three-dimensional dental laminate modification platform for adhering an ornament to a laminate fabricates a three-dimensional dental laminate and an ornament.

FIG. 8 is a diagram showing a hardware configuration of the server according to FIG. 1 by way of example.

It should be noted that throughout the drawings, like reference numbers are used to denote the same or similar elements, features and structures.

### DETAILED DESCRIPTIONS

The following embodiments are combinations of components and features of the embodiments in a predetermined form. Each component or feature may be optional unless explicitly stated otherwise. Each component or feature may be implemented in a non-combined manner with other components or features. Furthermore, various embodiments may be constructed by combining some components and/or features with each other. The order of operations as described in various embodiments may be changed. Some components or features of one embodiment may be included in other embodiments, or may be replaced with corresponding components or features of other embodiments.

In the description of the drawings, procedures or steps that may obscure the gist of various embodiments are not described, and procedures or steps that may be well-known to those skilled in the art are not described.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in this specification, specify the presence of the stated features, integers, operations, elements, and/or components, but do not preclude the presence or addition of one or more other features, integers, operations, elements, components, and/or portions thereof.

Furthermore, terms such as “unit”, and “module” as described herein may mean a unit that processes at least one function or operation, and may be implemented in a hardware or software manner, or in a combination of hardware and software.

Hereinafter, embodiments according to various embodiments will be described in detail with reference to the accompanying drawings. The detailed description set forth below in conjunction with the accompanying drawings is intended to illustrate illustrative embodiments of various embodiments, and is not intended to represent a single embodiment.

Furthermore, specific terms used in various embodiments are provided to help understanding of various embodiments, and these specific terms may be changed into other terms without departing from the technical spirit of various embodiments.

Hereinafter, a preferred embodiment according to the present disclosure will be described in detail with reference to the accompanying drawings.

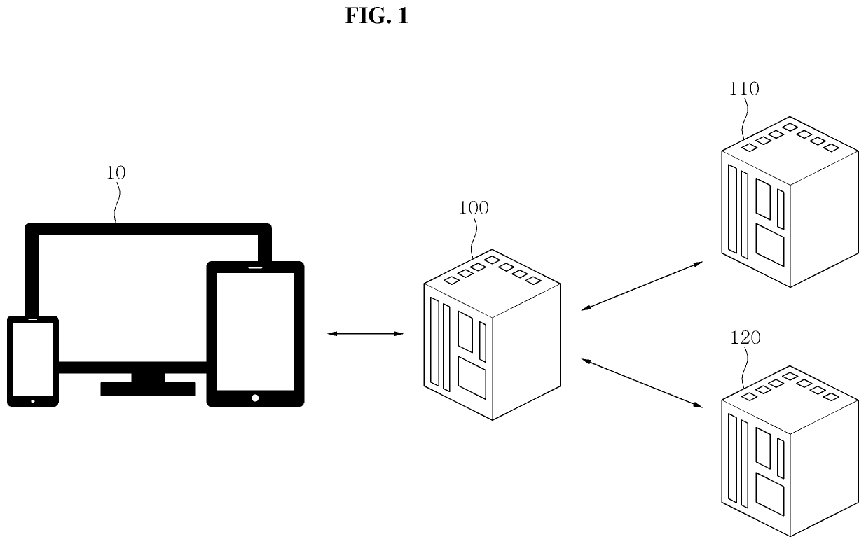

FIG. 1 is an illustrative view showing an operating environment of a server for operating a three-dimensional dental laminate modification platform for adhering ornaments thereto according to an embodiment of the present disclosure.

Referring to FIG. 1, a server 100 (hereinafter referred to as ‘server 100’) for operating a three-dimensional dental laminate modification platform for adhering ornaments thereto may receive a type of an ornament to be adhered to a three-dimensional dental laminate and, optionally, a position on the three-dimensional dental laminate at which the ornament is to be adhered thereto, from a web or an application installed in a user terminal 10. In this regard, the position on the three-dimensional dental laminate at which the ornament is to be adhered thereto may be directly selected by a user on the web or the application and then received from the web or the application, or may be a predetermined position. In this regard, the web or the application installed in the user terminal 10 may further provide a combined image (preview) in which the ornament has been adhered to a predetermined position of the three-dimensional dental laminate such that the user may pre-check the combined image. Furthermore, the web or the application installed in the user terminal 10 may retrieve pre-designed and stored ornaments from ornament database (DB) 120 as described later, and may provide the same to the user. Alternatively, the web or the application installed in the user terminal 10 may provide an interface on which the user may directly design a new ornament via the user terminal 10. In this regard, the interface may include at least one of functions of a conventional 3D program. Thus, a three-dimensional modeling file of the ornament designed via the 3D program may be generated and stored in the ornament database (DB) 120. Alternatively, the user may provide an object that the user wants to use as an ornament, and thus, a three-dimensional modeling file based on this object may be generated and stored in the ornament database (DB) 120. Then, the web or the application installed in the user terminal 10 may retrieve the three-dimensional modeling file from the DB and then may provide the same to the user. In this way, the user may design or select the ornament via various procedures and methods.

In this regard, the server 100 may receive a three-dimensional dental laminate modeling file stored in three-dimensional dental laminate database (DB) 110. In this regard, the received three-dimensional dental laminate modeling file may refer to a three-dimensional modeling file created so as to be registered on the user's teeth, and may be pre-created based on the user's teeth image. Furthermore, the three-dimensional dental laminate database (DB) 110 may be connected to the server 100 over a wired or wireless network. However, the present disclosure is not limited thereto, and the three-dimensional dental laminate database (DB) 110 may be implemented as one component of the server 100, and may be, for example, an internal storage device of the server.

In one example, each of the server 100 and the user terminal 10 may be embodied as any one of a desktop computer, a laptop computer, a notebook, a mobile phone, a smart phone, a tablet PC, a handheld phone, a smart watch, a smart glass, an e-book reader, PMP (portable multimedia player), a portable game console, a navigation device, a digital camera, a digital multimedia broadcasting (DMB) player, a digital audio recorder, a digital audio player, a digital video recorder, a digital video player, a personal digital assistant (PDA) the like. However, the present disclosure is not limited thereto.

Furthermore, the wired and wireless network schemes may include various communication schemes such as Bluetooth communication, Bluetooth Low Energy (BLE) communication, Near Field Communication (NFC), WLAN communication, Zigbee communication, Infrared Data Association (IrDA) communication, WFD (Wi-Fi Direct) communication, UWB (ultra-wideband) communication, Ant+ communication, WIFI communication, RFID (Radio Frequency Identification) communication, or mobile communication networks such as LTE or NR. However, the present disclosure is not limited thereto.

The server 100 may receive the type of the ornament to be adhered to the three-dimensional dental laminate and optionally, the position on the three-dimensional dental laminate at which the ornament is to be adhered thereto according to a request from the user terminal 10. In this regard, the position on the three-dimensional dental laminate at which the ornament is to be adhered thereto may be directly selected by a user on the web or the application and then received from the web or the application, or may be a predetermined position.

In this regard, the ornament to be adhered to the three-dimensional dental laminate may be implemented in various forms, such as jewelry including cubic or diamond, metal including gold and silver, an ornament implemented using luminescent material including noctilucence or UV reactive material, an additive having taste or scent, a text implemented in various fonts, a material having a color changing depending on temperature or moisture, etc. The present disclosure is not limited thereto, and the ornament may include ornaments implemented in various materials and shapes that may be adhered to the three-dimensional dental laminate and may not irreversibly damage the user's real teeth.

When the ornament to be adhered to the three-dimensional dental laminate has been selected according to the request from the user terminal 10, the server 100 may receive the three-dimensional modeling file of the ornament from the ornament database (DB) 120. In this regard, the received three-dimensional ornament modeling file may be pre-created based on the image of the ornament. Furthermore, the ornament database (DB) 120 may be connected to the server 100 over a wired or wireless network. However, the present disclosure is not limited thereto, and the DB 120 may be implemented as a component of the server 100, for example, an internal storage device of the server.

After the server 100 has received the three-dimensional dental laminate modeling file and the 3D ornament modeling file for the user according to the request from the user terminal 10, and optionally, the position on the three-dimensional dental laminate at which the ornament is to be adhered thereto, the server 100 may modify the three-dimensional dental laminate modeling file based on the 3D ornament modeling file.

For example, the server 100 may modify the three-dimensional dental laminate modeling file so that an adhered surface of the three-dimensional dental laminate and an adhered surface of the ornament are registered onto each other. In one example, when the adhered surface (e.g., rear surface) of the ornament adhered to the three-dimensional dental laminate is a flat surface without curvature, and the adhered surface of the three-dimensional dental laminate adhered to the ornament is a curved surface, and when there is no modification on the modeling file of the three-dimensional dental laminate or the ornament, the adhered surface of the three-dimensional dental laminate and the adhered surface of the ornament are not registered with each other, resulting in poor adhesive strength and easy separation therebetween. Further, the protruding ornament may cause injury to the user's oral cavity, and the insertion of foreign substances into between the adhered surfaces may deteriorate oral health.

Accordingly, in one example, the server 100 may form an intaglio having the same shape as that of the rear surface of the ornament by a predetermined depth on the adhered surface to which the ornament is adhered in the three-dimensional dental laminate modeling file to modify the adhered surface to the flat surface, such that the adhered surface of the three-dimensional dental laminate and the adhered surface of the ornament are registered onto each other. However, the present disclosure is not limited thereto, and the server 100 may modify the ornament modeling file to modify the rear surface of the ornament modeling file so as to have the same curved adhered surface as the curved adhered surface of the three-dimensional dental laminate, such that the adhered surface of the three-dimensional dental laminate and the adhered surface of the ornament are registered onto each other.

Furthermore, in another example, upon determination that a curvature or shape of the adhered surface to which the ornament is adhered in the three-dimensional dental laminate modeling file and a curvature or shape of the adhered surface to which the ornament is adhered to the three-dimensional dental laminate match each other, the server 100 may not modify the three-dimensional dental laminate modeling file. In this case, the server 100 may determine that the adhered surface of the three-dimensional dental laminate is registered with the adhered surface of the ornament.

In one example, when a surface area of the ornament is smaller than or equal to a predetermined percentage (e.g., 1/20) of an individual tooth area of the teeth to which the ornament is adhered in the three-dimensional dental laminate, the server 100 may determine not to modify the three-dimensional dental laminate modeling file.

Then, the server 100 may manufacture the ornament and the modified three-dimensional dental laminate using a 3D printer (e.g., Carbon 3D Printer by Google or Asiga 3D Printer) connected thereto over a wireless network. However, for smooth descriptions of the present disclosure, it is assumed that the server 100 is separately connected to the 3D printer in the manufacture of three-dimensional dental laminate. The present disclosure is not limited thereto, and the server 100 may include a function of the 3D printer or may communicate directly with the 3D printer.

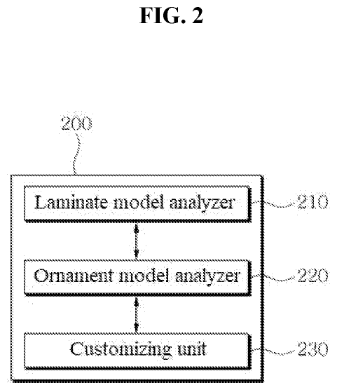

FIG. 2 is an illustrative diagram showing an example of a server for operating a three-dimensional dental laminate modification platform for adhering ornaments thereto.

Referring to FIG. 1 and FIG. 2, a server 200 (hereinafter referred to as ‘server 200’) for operating a three-dimensional dental laminate modification platform for adhering ornaments thereto may include a laminate model analyzer 210, an ornament model analyzer 220 and a customizing unit 230.

The laminate model analyzer 210 may receive a user's three-dimensional dental laminate modeling file according to a request from the user terminal 10.

In this regard, the three-dimensional dental laminate modeling file may be generated by irradiating light including infrared or short-wavelength laser to the laminate and then calculating a return time and a phase difference of the light, or may be generated based on a three-dimensional image or video including depth information up to an object included in an image and video which may be obtained using a stereo camera.

Furthermore, in an inner surface of the generated three-dimensional dental laminate modeling file, a shape of an outer surface of the user's tooth is formed in an intaglio manner. Thus, the inner surface of the three-dimensional dental laminate modeling file may be formed so as to be registered on the user's teeth. In this regard, the three-dimensional dental laminate modeling file may be a modeling file created by analyzing and scaling two-dimensional and three-dimensional images and videos as received via Computer Aided Design (CAD) (e.g., AutoCAD, Exocad and other dental CAD programs, etc.).

The laminate model analyzer 210 may analyze the received three-dimensional dental laminate modeling file and extract an area thereof onto which the ornament can be adhered. For example, the laminate model analyzer 210 may calculate an area size of an area corresponding to each of teeth in the received three-dimensional dental laminate modeling file. Next, the laminate model analyzer 210 may determine an area of a tooth having an area size larger than a predetermined area size as an area to which the ornament may be adhered, and thus may determine an area in the three-dimensional dental laminate modeling file to which the ornament may be adhered, based on the determined tooth.

In this regard, the laminate model analyzer 210 may segment the teeth image into each tooth image and identify an individual tooth in the received three-dimensional dental laminate modeling file using at least one algorithm among an image segmentation algorithm, a contour recognition algorithm (edge detection), a 3D surface analysis algorithm (surface curvature analysis or point cloud data processing), and a neural network analysis (CNN, 3D CNNs) algorithm.

Furthermore, the laminate model analyzer 210 may calculate a curvature of a surface of each tooth in the received three-dimensional dental laminate modeling file. For example, the laminate model analyzer 210 may identify a plurality of teeth of the three-dimensional dental laminate modeling file and calculate a curvature of an outer surface of each of the identified teeth. In this regard, the laminate model analyzer 210 may calculate the curvature of the outer surface at the center of gravity of each tooth. In one example, the curvature of the outer surface of the canine tooth may be calculated to be greater than the curvature of the outer surface of each of the incisors. The larger the curvature of the tooth, the more difficult it is to attach the ornament to the outer surface thereof, or the deeper the depth of the intaglio having the same shape as that of the ornament and formed such that the ornament can be correctly registered on the tooth.

After the curvature has been calculated, the laminate model analyzer 210 may determine a tooth having a curvature smaller than a predetermined curvature among the teeth identified in the three-dimensional dental laminate modeling file as a tooth to which the ornament can be adhered. For example, when the radius of curvature of the incisor is 10 mm, the radius of curvature of the canine is 7 mm, and the predetermined radius of curvature is 9 mm, the laminate model analyzer 210 may determine the incisor having a radius of curvature greater than the predetermined radius of curvature (where the curvature may be defined as a reciprocal of the radius of curvature) as a tooth to which the ornament can be adhered, and may determine the canine having a radius of curvature smaller than the predetermined radius of curvature as a tooth to which the ornament cannot be adhered.

The ornament model analyzer 220 may receive the ornament modeling file according to a request from the user terminal 10.

For example, the ornament modeling file may be pre-created for each ornament and may be pre-stored in the ornament database (DB) 120. Furthermore, the present disclosure is not limited thereto. The ornament modeling file may be generated by irradiating light including infrared or short-wavelength laser to the ornament and then calculating a return time and a phase difference of the light, or may be generated based on a three-dimensional image or video including depth information up to an object included in an image and video which may be obtained using a stereo camera.

Furthermore, the ornament modeling file may be a modeling file created by analyzing and scaling two-dimensional and three-dimensional images and videos as received via Computer Aided Design (CAD) (e.g., AutoCAD, Exocad and other dental CAD programs, etc.).

In one example, the ornament model analyzer 220 may generate a surface image of an adhered surface (e.g., rear surface) of the ornament to which the three-dimensional laminate is adhered. For example, the ornament model analyzer 220 may determine the adhered surface (e.g., rear surface) of the ornament adhered to the three-dimensional laminate in the ornament three-dimensional modeling file, and may extract an image of the determined adhered surface therefrom. Furthermore, when the adhered surface of the ornament has been determined, the ornament model analyzer 220 may extract a thickness of the ornament in a normal direction to the adhered surface of the ornament as height information. Thus, the ornament model analyzer 220 may provide customizing information so that when the ornament is adhered to the three-dimensional laminate, the ornament is adhered thereto based on the determined adhered surface and the height information.

The customizing unit 230 may modify the adhered surface to which the ornament is adhered in the three-dimensional dental laminate modeling file based on an extracted image of the adhered surface of the ornament, such that the three-dimensional dental laminate modeling file may be customized so that the ornament and the three-dimensional dental laminate are registered onto each other.

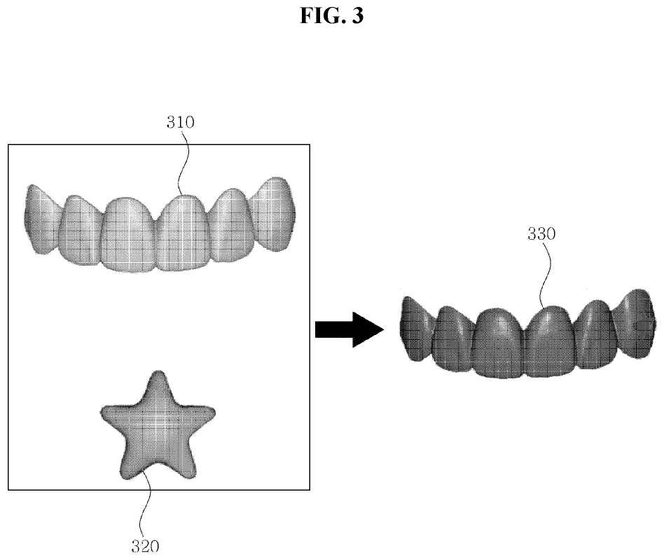

FIG. 3 is an illustrative view to illustrate an example of customizing a three-dimensional dental laminate modeling file based on a shape of the ornament.

Referring to FIG. 2 and FIG. 3, the customizing unit 230 may generate a customized three-dimensional laminate modeling file 330 based on the analysis result of a three-dimensional laminate modeling file 310 and an ornament modeling file 320. In one example, the customizing unit 230 may modify the ornament modeling file 320 by adjusting a size of the ornament to an area size smaller than a size of an area of each divided tooth of the teeth of the three-dimensional dental laminate modeling file as calculated by the laminate model analyzer 210.

Next, the customizing unit 230 may form an intaglio in a position to which the ornament is to be adhered in the three-dimensional dental laminate modeling file, based on the surface image of the adhered surface of the ornament generated via the ornament model analyzer 220. In this regard, the depth of the intaglio may be pre-determined based on the height information as the thickness of the ornament in the direction normal to the adhered surface extracted using the ornament model analyzer 220. For example, the depth of the formed intaglio may be a predetermined percentage of the height information. For example, when the height information (thickness) of the ornament is 3 mm, the customizing unit 230 may form the intaglio having a depth of 1 mm, which is a predetermined percentage (e.g., ⅓) of the height information (thickness) of the ornament.

However, the present disclosure is not limited thereto, and the customizing unit 230 may modify the ornament modeling file based on the curvature of the surface of each tooth in the three-dimensional dental laminate modeling file calculated by the laminate model analyzer 210. In one example, the customizing unit 230 may customize the ornament modeling file by modifying the adhered surface extracted from the ornament modeling file so as to have a curvature of the three-dimensional dental laminate at a position thereof where the ornament is to be adhered thereto in the three-dimensional dental laminate modeling file. In other words, the customizing unit 230 may modify the inner surface of the ornament in the ornament modeling file, based on the calculated curvature of the outer surface of each tooth so that the curvature of the adhered surface thereof is the same as the curvature of the outer surface of each tooth at the position thereof at which the ornament is to be adhered thereto in the three-dimensional dental laminate modeling file. Furthermore, in another embodiment, upon determination that a curvature or shape of the adhered surface to which the ornament is adhered in the three-dimensional dental laminate modeling file and a curvature or shape of the adhered surface to which the ornament is adhered to the three-dimensional dental laminate match each other, the customizing unit 230 may not modify the three-dimensional dental laminate modeling file. In this case, the customizing unit 230 may determine that the adhered surface of the three-dimensional dental laminate is registered with the adhered surface of the ornament. The customizing unit 230 may modify the ornament modeling file by modifying only the height of the ornament adhered to the three-dimensional dental laminate based on the height information as the thickness of the ornament in the normal direction to the adhered surface of the ornament extracted using the ornament model analyzer 220.

Further, when a surface area of the ornament is smaller than or equal to a predetermined percentage (e.g., 1/20) of an individual tooth area of the teeth to which the ornament is adhered in the three-dimensional dental laminate, the customizing unit 230 may determine not to modify the three-dimensional dental laminate modeling file.

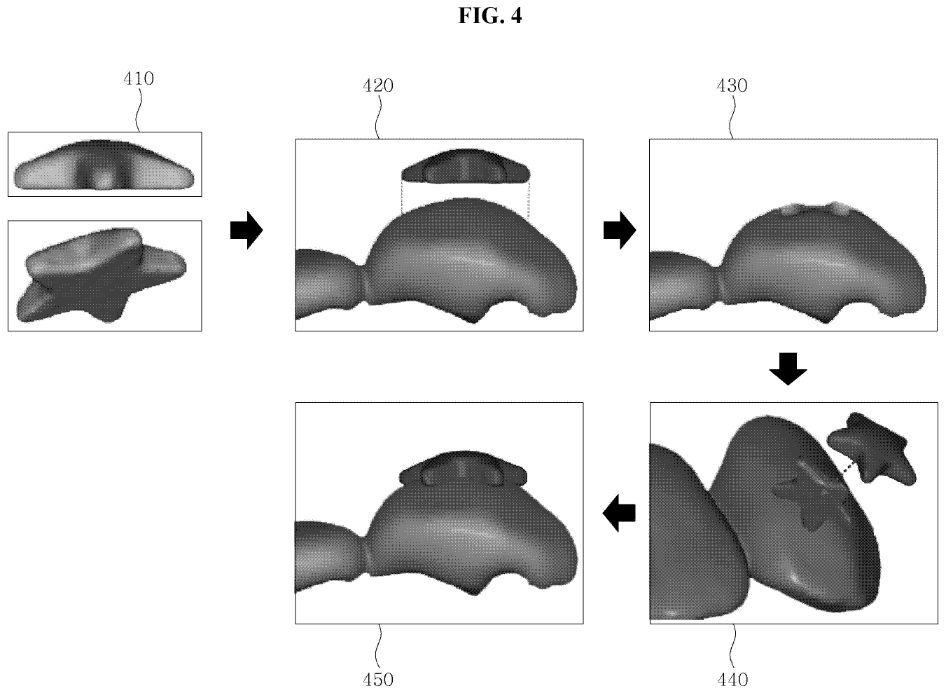

FIG. 4 is a detailed illustrative diagram for illustrating customization of a modeling file according to an embodiment of the present disclosure.

Referring to FIG. 2 and FIG. 4, the ornament model analyzer 220 may analyze the received ornament modeling file, and extract an image 410 of the adhered surface of the ornament adhered to the three-dimensional dental laminate. Furthermore, the laminate model analyzer 210 may analyze the received three-dimensional dental laminate modeling file, identify each of the teeth, and calculate a curvature 420 of each of the teeth to which the ornament is adhered. In this regard, the customizing unit 230 may modify the three-dimensional dental laminate modeling file based on the surface image 410 of the ornament analyzed by the ornament model analyzer 220. Specifically, the customizing unit 230 may form an intaglio 430 having the same shape as that of the ornament's surface image in the adhered surface of the three-dimensional dental laminate to which the ornament is to be adhered. In this regard, the depth of the intaglio formed on the three-dimensional dental laminate may be pre-determined based on the height information calculated as the thickness of the ornament in the normal direction to the adhered surface of the ornament adhered to the three-dimensional dental laminate. However, the present disclosure is not limited thereto. The depth may be determined based on various variables. For example, when the height of the ornament is 3 mm and the predetermined depth of the intaglio is ⅓ of the height of the ornament, the depth of the intaglio to be formed may be determined as 3 mm*⅓=1 mm. According to another example, the depth may be determined based on the thickness of the ornament.

The customizing unit 230 may combine the modified three-dimensional dental laminate modeling file with the ornament modeling file 440 and may determine whether the three-dimensional dental laminate is correctly registered with the ornament, based on the combination. Furthermore, the customizing unit 230 may provide an image 450 (preview) in which the ornament is combined with the three-dimensional dental laminate. Thus, the user may identify a structure in which the ornament is adhered to the three-dimensional dental laminate, based on the preview.

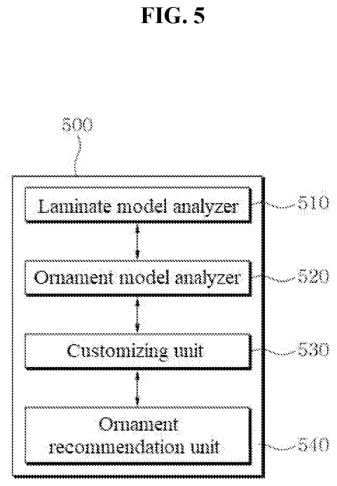

FIG. 5 is an illustrative diagram showing another example of a server for operating a three-dimensional dental laminate modification platform for adhering ornaments thereto. Referring to FIG. 5, a server 500 (hereinafter referred to as ‘server 500’) for operating the three-dimensional dental laminate modification platform for adhering ornaments thereto may include a laminate model analyzer 510, an ornament model analyzer 520, a customizing unit 530, and an ornament recommendation unit 540. In this regard, the laminate model analyzer 510), the ornament model analyzer 520, and the customizing unit (530) may be identical with the laminate model analyzer 210, the ornament model analyzer 220 and the customizing unit 230, respectively. Thus, a detailed description thereof may be omitted.

The ornament recommendation unit 540 may recommend the ornament that can be adhered to the three-dimensional dental laminate based on the analysis result of the laminate model analyzer 510.

For example, the ornament recommendation unit 540 may receive three-dimensional dental laminate information and ornament information. In this regard, the three-dimensional dental laminate information may include a radius of curvature (mm), a laminate thickness (mm), an interproximal space (mm), a surface area of each tooth (mm²), coefficient of thermal expansion (° C.⁻¹), transparency, an adhesive interface strength (MPa), water absorption, and flexural strength (MPa).

Furthermore, the ornamental information may include an adhesion size (mm³), an adhesion weight (g), thermal conductivity (W/m K), hardness (Mohs or Vickers), flexibility (GPa), reflectance, color stability (ΔE), adhesion strength (MPa), bacterial adhesion (cfu/cm²), thermal expansion coefficient (° C.⁻¹). However, the present disclosure is not limited thereto.

The ornament recommendation unit 540 may classify the received three-dimensional dental laminate information into at least one category based on attributes. For example, the ornament recommendation unit 540 may classify the radius of curvature, the laminate thickness, the interproximal space, the surface area of each tooth, and the coefficient of thermal expansion in the received three-dimensional dental laminate information into a physical property category, and may classify the transparency, the adhesive interface strength and the flexural strength in the received three-dimensional dental laminate information into adhesion index.

Furthermore, the ornament recommendation unit 540 may classify the adhesion size, the adhesion weight, the thermal conductivity, the hardness, the flexibility and the thermal expansion coefficient in the received ornament information into the physical property category, and may classify the reflectance and the color stability in the received ornament information into optical properties, and may classify the adhesion strength and the bacterial adhesion in the received ornament information into the adhesion index.

After the received three-dimensional dental laminate information has been classified into each of the categories, the ornament recommendation unit 540 may combine the information included in each of the categories with each other to calculate a dental laminate score.

For example, the ornament recommendation unit 540 may calculate a laminate physical property score P based on the radius of curvature R, the laminate thickness T, the interproximal space, the surface area A of each tooth, and the thermal expansion coefficient C belonging to the physical property category in the three-dimensional dental laminate information and according to a following Mathematical Equation:

$$P = \frac{T \times A \times I}{R \times C} \qquad [ Mathematical Equation ]$$

Specifically, the radius of curvature R represents the curvature of the tooth. The larger the radius of curvature, the flatter the tooth surface, whereas the smaller the radius of curvature, the more curved the tooth surface. Furthermore, regarding the laminate thickness T, as the laminate thickness is larger, a more rigid ornament can be adhered to the laminate, and the adhered surface may have rigidity. Furthermore, the surface area A of each tooth represents a size of an area of each tooth by which the ornament can be adhered to each tooth. A larger surface area A may mean that a size of an area of each tooth by which the ornament can be adhered to each tooth is larger or the number of the ornaments which can be adhered to each tooth is larger. Furthermore, the interproximal space I may refer to a spacing between adjacent teeth required to prevent the three-dimensional dental laminate and the ornament from interfering with a tooth adjacent thereto. Furthermore, the coefficient of thermal expansion C represents a volume by which the laminate expands or contracts as the temperature changes. The larger the coefficient of thermal expansion, the larger the deformation of the laminate as the temperature changes.

In this regard, the numerator factor, that is, the laminate thickness T, the surface area A of each tooth, and the interproximal space I may mean the volume and the space that may be used with respect to the ornament. The denominator factor, that is, the radius of curvature R and the thermal expansion coefficient C may serve as a correction and scaling factor indicating adaptability to the curvature of the tooth and the temperature change.

For example, under assumption that the radius of curvature R=14 mm, the laminate thickness T=0.6 mm, the interproximal space I=2 mm, the surface area A of each tooth=65 mm² and the thermal expansion coefficient C=10×10⁻⁶° C.⁻¹ (average thermal expansion coefficient of a dental material) as the physical properties of a specific tooth of the three-dimensional dental laminate, the laminate physical property score P is calculated as follows:

$$P = \frac{0.6 \times 65 \times 2}{14 \times 10 \times 1 {0}^{- 6}} = 5460 \qquad [ Mathematical Equation ]$$

In one example, the ornament recommendation unit 540 may normalize the laminate physical property score P via min-max normalization. For example, when Pₘᵢₙ is 1000 and Pₘₐₓ is 15000, a normalized laminate physical property score Pₙₒₘ is calculated based on a following Mathematical Equation:

$${P}_{n o m} = \frac{5460 - 1000}{15000 - 1000} = 0.32 \qquad [ Mathematical Equation ]$$

In other words, the calculated physical property score Pₙₒₘ is used as an index indicating a value by which the laminate is suitable for the ornament in consideration of constraints caused by the curvature of the tooth and the physical property of the laminate material. The closer the physical property score Pₙₒₘ is to 0, the more difficult it is to adhere the ornament to the laminate. The closer the physical property score Pₙₒₘ is to 1, the ornament may be selected in a wider range.

For example, the ornament recommendation unit 540 may recommend an ornament based on a comparing result of the calculated physical property score Pₙₒₘ with a predetermined threshold value. In one example, when the physical property score Pₙₒₘ is in a range of 0 inclusive to 0.2 exclusive, the ornament recommendation unit 540 may recommend the ornament and the metal ornament having a thermal expansion coefficient within a predetermined percentage range (e.g., 10%) of that of the dental laminate, and having an area size smaller than a predetermined percentage range (e.g., 20%) of the surface area size of each tooth. Furthermore, when the physical property score Pₙₒₘ is in a range of 0.2 inclusive to 0.8 exclusive, this means that flexibility and the rigidity are in balance with each other. Thus, the ornament recommendation unit 540 may recommend the ornament having an area size of a predetermined percentage range (e.g., 60%) of the surface area size of each tooth. When the physical property score Pₙₒₘ is in a range of 0.8 inclusive to 1 inclusive, the ornament recommendation unit 540 may recommend the ornament having an area size of a predetermined percentage range (e.g., 90%) of the surface area size of each tooth.

Furthermore, the ornament recommendation unit 540 may calculate a laminate adhesion index score AdI based on the transparency Tr, the adhesive interface strength Ai, and the flexural strength Fs included in the adhesion index category in the three-dimensional dental laminate information, and according to a following Mathematical Equation:

$$AdI = Tr \times \frac{A i + F s}{2} \qquad [ Mathematical Equation ]$$

That is, the calculated adhesion index score AdI may be calculated by applying the transparency as a weight to an average of the adhesive interface strength and the flexural strength.

Specifically, the transparency Tr has a value of 0 to 1, where 0 means being opaque and 1 means being transparent. When the laminate is transparent, the ornaments that may emphasize the transparency, such as metals and jewels, may be suitable for the laminate. Furthermore, the adhesive interface strength Ai indicates how well the ornament may be adhered to the laminate and has a unit of MPa. As the adhesive interface strength is larger, the laminate may support the larger and heavier ornament. Furthermore, the flexural strength Fs refers to an ability by which the three-dimensional dental laminate may withstand the stress when the laminate is bent, and is measured in MPa. As the flexural strength of the laminate is higher, the laminate may fill an entire area of each tooth or may withstand a flat ornament.

In one example, when the transparency Tr=0.9, the adhesive interface strength Ai=20 MPa, and the flexural strength Fs=30 MPa, the adhesion index score AdI is calculated according to a following Mathematical Equation:

$$AdI = 0.9 \times \frac{20 + 30}{2} = 22.5 \qquad [ Mathematical Equation ]$$

In one example, the ornament recommendation unit 540 may normalize the laminate adhesion index score (AdI) via min-max normalization. For example, when AdIₘᵢₙ is 5 and AdIₘₐₓ is 35, the normalized laminate adhesion index score AdIₙₒₘ is calculated according to a following Mathematical Equation:

$${AdI}_{n o m} = \frac{22.5 - 5}{35 - 5} = 0.6 \qquad [ Mathematical Equation ]$$

The ornament recommendation unit 540 may determine whether the calculated adhesion index score AdIₙₒₘ is within a predetermined range and recommend the ornament based on the determination result. For example, when the adhesion index score AdIₙₒₘ is in a range of 0 inclusive to 0.3 exclusive, the ornament recommendation unit 540 may recommend an opaque ornament with high adhesion. When the adhesion index score AdIₙₒₘ is in a range of 0.3 inclusive to 0.7 exclusive, the ornament recommendation unit 540 may recommend an ornament having middle level adhesion and transparency. When the adhesion index score AdIₙₒₘ is in a range of 0.7 inclusive to 1 inclusive, the ornament recommendation unit 540 may recommend a transparent ornament with low adhesive strength.

Furthermore, after the dental laminate physical property score Pₙₒₘ and the adhesion index score A_{dInom} have been calculated, the ornament recommendation unit 540 may calculate a dental laminate score DLS based on an average of the physical property score Pₙₒₘ and the adhesion index score AdIₙₒₘ, according to a following Mathematical Equation. For example, it is assumed that the dental laminate physical property score Pₙₒₘ is 0.8 and the adhesion index score AdIₙₒₘ is 0.65:

$$D L S = \frac{{P}_{n o m} + {AdI}_{n o m}}{2} = \frac{0.8 + 0.65}{2} = 0.725 \qquad [ Mathematical Equation ]$$

The ornament recommendation unit 540 may recommend an ornament based on the calculated DLS. For example, the ornament recommendation unit 540 may recommend an ornament based on a determining result of whether the calculated DLS is in a predetermined score range. In one example, when the DLS is in a range of 0 inclusive to 0.5 exclusive, the ornament recommendation unit 540 may recommend a base ornament. The base ornament may refer to an ornament having a weight in a range of 20% from a lowest value, a thermal conductivity in a range of 20% from a lowest value, and an adhesive interface strength in a range of 20% from a highest value, based on the ornament information. Furthermore, when the DLS is in a range of 0.5 inclusive to 0.8 exclusive, the ornament recommendation unit may recommend an advanced ornament. The advanced ornament may refer to an ornament having weight in a range of 20% to 80% from a lowest value, a thermal conductivity in a range of 20% to 80% from a lowest value, and an adhesive interface strength in a range of 30% from a highest value, based on the ornament information.

Furthermore, when the DLS is in a range of 0.8 exclusive to 1 inclusive, the ornament recommendation unit may recommend a premium ornament. The premium ornament may refer to an ornament having weight in a range of 80% to 100% from a lowest value, a thermal conductivity in a range of 80% to 100% from a lowest value, and an adhesive interface strength in a range of 80% from a highest value, based on the ornament information.

That is, the ornament recommendation unit 540 may recommend the ornaments with the larger weight and the higher thermal conductivity and the lower adhesive strength as the DLS is higher. Thus, the ornament recommendation unit 540 may recommend more diverse ornaments.

In one example, after the received ornament information have been classified into each of the categories, the ornament recommendation unit 540 may combine the information included in each category with each other to calculate an ornament score.

For example, the ornament recommendation unit 540 may calculate an ornament physical property score OPS based on the adhesion size AdS, the adhesion weight Aw, the thermal conductivity Tc, the hardness H, the flexibility F, and the thermal expansion coefficient Te included in the physical property category in the ornament information and according to a following Mathematical Equation:

$$O P S = \frac{A d S \times F \times ( 10 - H )}{A w \times T c \times T e} \qquad [ Mathematical Equation ]$$

Specifically, the adhesion size AdS refers to the surface area of the ornament in contact with the laminate, and a larger contact surface area may indicate stable adhesion. The higher the value of adhesion weight Aw, the greater the pressure and discomfort applied to the tooth of interest and surrounding teeth. As the thermal conductivity Tc is lower, the ornament may not transfer temperature change to the laminate rapidly and thus the laminate may not be affected by rapid temperature change. Furthermore, the hardness H is based on Mohs's hardness, and the lower the hardness, the less likely the ornament may cause injury to the gum adjacent thereto and oral cavity. The flexibility F refers to be an indicator of how accurately the ornament may be registered on the three-dimensional dental laminate, and the greater the flexibility, the greater the adhesion. Furthermore, as the coefficient of thermal expansion Te is more approximate to that of the three-dimensional dental laminate, consistent registration may be guaranteed under the temperature changes.

In one example, when the adhesion size AdS=20 mm², the adhesion weight Aw=0.5 g, the thermal conductivity Tc=15 W/mK, the hardness H=7, the flexibility F=0.7 and the thermal expansion coefficient Te=0.000015 1/° C., the ornament physical property score OPS is calculated according to a following Mathematical Equation:

$$O P S = \frac{20 \times 0.7 \times ( 10 - 7 )}{0.5 \times 15 \times 0.000015} = 186.67 \qquad [ Mathematical Equation ]$$

In one example, the ornament recommendation unit 540 may normalize the ornament physical property score OPS via min-max normalization. For example, when OPSₘᵢₙ is 50 and OPSₘₐₓ is 500, the normalized ornament physical property score OPSₙₒₘ is calculated according to a following Mathematical Equation:

$${OPS}_{n o m} = \frac{186.67 - 50}{500 - 50} = 0.29 \qquad [ Mathematical Equation ]$$

That is, the normalized ornament physical property score OPSₙₒₘ has a value of 0 to 1. As the normalized ornament physical property score OPSₙₒₘ is closer to 0, the ornament is lighter, more flexible, and has a lower thermal conductivity. Therefore, the calculated ornament physical property score OPSₙₒₘ of 0.29 is a relatively low score, which may mean that the ornament is light, flexible, and has low thermal conductivity.

In one example, the ornament recommendation unit 540 may classify the ornament into an ornament grade set to a predetermined score range according to the ornament physical property score OPSₙₒₘ. In one example, the ornament recommendation unit 540 classifies the ornament as a base ornament (BO) when the calculated ornament physical property score OPSₙₒₘ is in a range of 0.66 inclusive to 1 inclusive. When the calculated ornament physical property score OPSₙₒₘ is in a range of 0.33 inclusive to 0.66 exclusive, the ornament recommendation unit 540 classifies the ornament as an advanced ornament (AO). When the calculated ornament physical property score OPSₙₒₘ is in a range of 0 inclusive to 0.33 exclusive, the ornament recommendation unit 540 classifies the ornament as a premium ornament (PA).

Accordingly, the ornament recommendation unit 540 may recommend an ornament having the ornament grade classified based on the ornament physical property score OPSₙₒₘ and a recommended ornament grade classified based on the dental laminate score DLS equal to each other as an ornament suitable for the three-dimensional dental laminate.

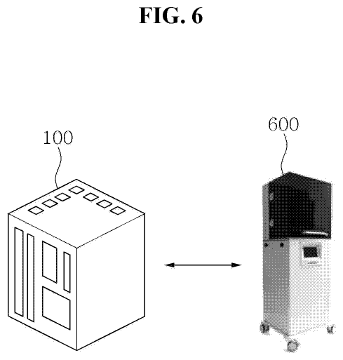

FIG. 6 is an illustrative view to illustrate an example of manufacturing a three-dimensional dental laminate and ornament.

Referring to FIG. 1 and FIG. 6, the server 100 may manufacture the ornament and the three-dimensional dental laminate using a 3D printer (e.g., Carbon 3D Printer by Google or Asiga 3D Printer) 600 connected thereto over a wireless network.

However, for smooth descriptions of the present disclosure, it is assumed that the server 100 is separately connected to the 3D printer 600 in the manufacture of the three-dimensional dental laminate. The present disclosure is not limited thereto, and the server 100 may include a function of the 3D printer 600 or may communicate directly with the 3D printer.

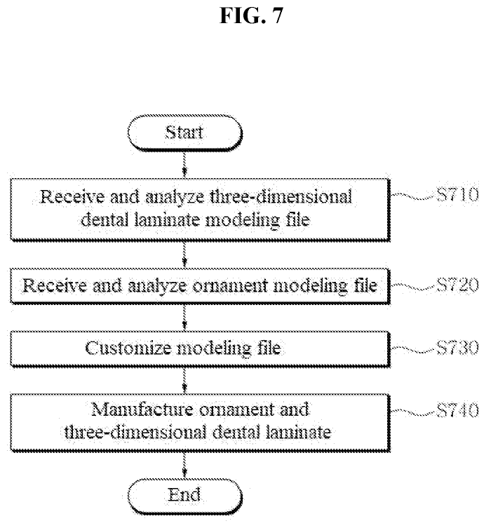

FIG. 7 is an illustrative flow chart to illustrate a method in which a server for operating a three-dimensional dental laminate modification platform for adhering an ornament to the laminate fabricates a three-dimensional dental laminate and ornament.

Referring to FIG. 1 and FIG. 7, the server 100 may receive and analyze the three-dimensional dental laminate modeling file for the user according to the request from the user terminal 10 in S710.

Then, the server 100 may select an ornament to be adhered to the three-dimensional dental laminate according to the request from the user terminal 10. Furthermore, when the ornament to be adhered to the three-dimensional dental laminate has been selected according to the request from the user terminal 10, the server 100 may receive and analyze the three-dimensional modeling file of the ornament in S720.

Then, after receiving the three-dimensional dental laminate modeling file and the ornament modeling file for the user according to the request from the user terminal 10, the server 100 may customize the three-dimensional dental laminate modeling file in S730.

For example, the server 100 may modify the three-dimensional dental laminate modeling file so that the adhered surface of the three-dimensional dental laminate and the adhered surface of the ornament are registered on each other.

In one example, the server 100 may form an intaglio in the surface of the laminate to which the ornament is to be adhered in the three-dimensional dental laminate modeling file such that the adhered surface of the three-dimensional dental laminate and the adhered surface of the ornament are registered on each other. However, the present disclosure is not limited thereto, and the server 100 may modify the ornament modeling file such that the adhered surface of the ornament has the same curvature as that of the adhered surface of the three-dimensional dental laminate and thus, the adhered surface of the three-dimensional dental laminate and the adhered surface of the ornament are registered on each other.

Then, the server 100 may manufacture the ornament and the three-dimensional dental laminate using the 3D printer (e.g., Carbon 3D Printer by Google or Asiga 3D Printer) connected thereto over a wireless network, based on the modified modeling file in S740.

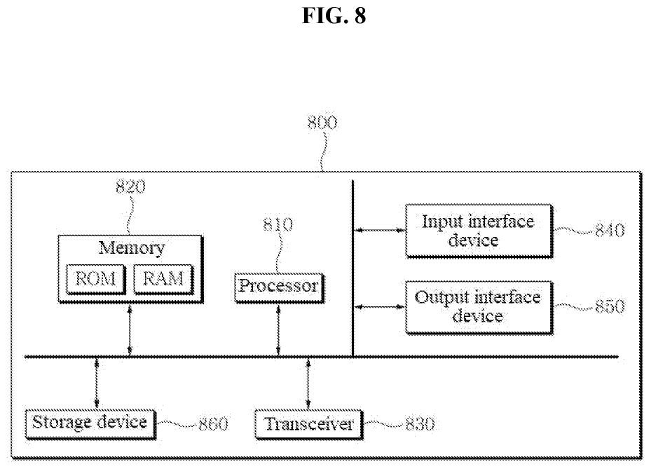

FIG. 8 is a diagram showing a hardware configuration of the server according to FIG. 1 by way of example.

Referring to FIG. 8, a server 800 for operating a three-dimensional dental laminate modification platform for adhering ornaments thereto may include at least one processor 810 and a memory 820 that stores therein instructions. When the instructions are executed by the at least one processor 810, the instructions cause the at least one processor 810 to perform at least one operation.

In this regard, the at least one processor 810 may be embodied as a central processing unit (CPU), a graphics processing unit (GPU), or a dedicated processor for performing operations of the method according to embodiments of the present disclosure. Each of the memory 820 and a storage device 860 may be embodied as at least one of a volatile storage medium or a non-volatile storage medium. For example, the memory 820 may be embodied as at least one of read only memory (ROM) or random access memory (RAM).

Furthermore, the server 800 for operating the three-dimensional dental laminate modification platform for adhering ornaments thereto may include a transceiver 830 which performs communication over a wireless network. Furthermore, the server 800 for operating the three-dimensional dental laminate modification platform for adhering ornaments thereto may further include an input interface device 840, an output interface device 850, the storage device 860, etc. The above components included in the server 800 for operating the three-dimensional dental laminate modification platform for adhering ornaments thereto may be connected to each other via a bus 870 and may communicate with each other via the bus.

The methods according to the present disclosure may be implemented in a form of program instructions that may be executed by various computer means and may be recorded in a computer-readable medium. The computer-readable media may include program instructions, data files, data structures, etc. alone or in combination with each other. The program instructions recorded in the computer-readable medium may be specially designed and configured to be adapted to the present disclosure, or may be known to those skilled in computer software.

Examples of the computer-readable media may include a hardware device specially configured to store therein and execute the program instructions, such as ROM, RAM, and flash memory. Examples of the program instructions may be composed of not only machine language codes generated by a compiler but also high-level language codes that may be executed by a computer using an interpreter or the like. The above-described hardware device may be configured to operate as at least one software module to perform the operation of the present disclosure, or vice versa.

Furthermore, the above-described method or device may be implemented such that all or some of components or functions thereof are combined with each other, or may be implemented such that all or some of components or functions thereof are separated from each other.

Although the present disclosure has been described with reference to preferred embodiments of the present disclosure, it may be appreciated that those skilled in the art may make modifications and changes to the present disclosure within the scope not departing from the spirit and field of the present disclosure as described in the following claims.

What is claimed is:

1. A server for operating a three-dimensional dental laminate modification platform for adhering an ornament to a laminate, the server comprises:
at least one processor, and a memory operably connected to the at least one processor,
wherein the memory stores therein instructions,
wherein when the instructions are executed by the at least one processor, the instructions cause the at least one processor to:
receive a three-dimensional dental laminate modeling file;
receive an ornament modeling file;
determine an adhered surface of the ornament to be adhered to the three-dimensional laminate in the received ornament modeling file, and extract an image of the adhered surface therefrom; and
upon determination that the three-dimensional dental laminate modeling file is required to be modified, form an intaglio based on the extracted image of the adhered surface in a position on the laminate in which the ornament is to be adhered thereto in the three-dimensional dental laminate modeling file, thereby modifying the three-dimensional dental laminate modeling file.

2. The server of claim 1, wherein the instructions are executed by the at least one processor, the instructions further cause the at least one processor to:
extract, as height information, a thickness of the ornament in a normal direction to the adhered surface thereof; and
determine a depth of the formed intaglio as a predetermined percentage of the height information.

3. The server of claim 1, wherein when the instructions are executed by the at least one processor, the instructions further cause the at least one processor to:
identify a plurality of teeth included in the received three-dimensional dental laminate modeling file;
calculate a curvature of an outer surface of each of the identified plurality of teeth; and
determine a tooth having the calculated curvature smaller than a predetermined curvature as a tooth to which the ornament can be adhered.

4. The server of claim 3, wherein when the instructions are executed by the at least one processor, the instructions further cause the at least one processor to modify the ornament modeling file such that a curvature of the adhered surface of the ornament is equal to a curvature of a tooth at a position on the three-dimensional dental laminate at which the ornament is adhered to the laminate.

5. The server of claim 1, wherein when the instructions are executed by the at least one processor, the instructions further cause the at least one processor to receive a position on the three-dimensional dental laminate at which the ornament is to be adhered to the laminate.

6. The server of claim 1, wherein when the instructions are executed by the at least one processor, the instructions further cause the at least one processor to:
receive three-dimensional dental laminate information;
classify the received three-dimensional dental laminate information into at least one category based on attributes thereof;
combine the three-dimensional dental laminate information included in each classified category with each other to calculate a dental laminate score; and
determine a recommended ornament grade based on the calculated dental laminate score and recommend the ornament based on the grade.

7. The server of claim 1, wherein when the instructions are executed by the at least one processor, the instructions further cause the at least one processor to:
receive ornament information;
classify the received ornament information into at least one category based on attributes thereof;
combine the ornament information included in each classified category with each other to calculate an ornament physical property score; and
classify the ornament into an ornament grade set to a predetermined score range, based on the calculated ornament physical property score.

8. The server of claim 7, wherein when the instructions are executed by the at least one processor, the instructions further cause the at least one processor to recommend an ornament having the ornament grade classified based on the calculated ornament physical property score and the recommended ornament grade classified based on the dental laminate score equal to each other as an ornament suitable for the three-dimensional dental laminate.

9. The server of claim 7, wherein the calculating of the ornament physical property score includes:
dividing a multiplication of an adhesion size, flexibility and hardness of the ornament by a multiplication of an adhesion weight, thermal conductivity, and thermal expansion coefficient of the ornament to obtain the ornament physical property score; and
performing minimum-maximum normalization based on a predetermined minimum ornament physical property score and a predetermined maximum ornament physical property score to obtain a normalized ornament physical property score.

10. A method for modifying a three-dimensional dental laminate for adhering an ornament to the laminate, wherein the method is carried out in a server for operating a three-dimensional dental laminate modification platform for adhering the ornament to the laminate,
wherein the method comprises:
receiving a three-dimensional dental laminate modeling file;
receiving an ornament modeling file;
determining an adhered surface of the ornament to be adhered to the three-dimensional laminate in the received ornament modeling file, and extracting an image of the adhered surface therefrom; and
upon determination that the three-dimensional dental laminate modeling file is required to be modified, forming an intaglio based on the extracted image of the adhered surface in a position on the laminate in which the ornament is to be adhered thereto in the three-dimensional dental laminate modeling file, thereby modifying the three-dimensional dental laminate modeling file.

11. The method of claim 10, further comprising:
extracting, as height information, a thickness of the ornament in a normal direction to the adhered surface thereof; and
determining a depth of the formed intaglio as a predetermined percentage of the height information.

12. The method of claim 10, further comprising:
identifying a plurality of teeth included in the received three-dimensional dental laminate modeling file;
calculating a curvature of an outer surface of each of the identified plurality of teeth; and
determining a tooth having the calculated curvature smaller than a predetermined curvature as a tooth to which the ornament can be adhered.

13. The method of claim 12, further comprising modifying the ornament modeling file such that a curvature of the adhered surface of the ornament is equal to a curvature of a tooth at a position on the three-dimensional dental laminate at which the ornament is to be adhered to the laminate.

14. The method of claim 10, further comprising receiving a position on the three-dimensional dental laminate at which the ornament is to be adhered to the laminate.

15. The method of claim 10, further comprising:
receiving three-dimensional dental laminate information;
classifying the received three-dimensional dental laminate information into at least one category based on attributes thereof;
combining the three-dimensional dental laminate information included in each classified category with each other to calculate a dental laminate score; and
determining a recommended ornament grade based on the calculated dental laminate score and recommending the ornament based on the grade.

16. The method of claim 10, further comprising:
receiving ornament information;
classifying the received ornament information into at least one category based on attributes thereof;
combining the ornament information included in each classified category with each other to calculate an ornament physical property score; and
classifying the ornament into an ornament grade set to a predetermined score range, based on the calculated ornament physical property score.

17. The method of claim 16, further comprising recommending an ornament having the ornament grade classified based on the calculated ornament physical property score and the recommended ornament grade classified based on the dental laminate score equal to each other as an ornament suitable for the three-dimensional dental laminate.

18. The method of claim 16, wherein the calculating of the ornament physical property score includes:
dividing a multiplication of an adhesion size, flexibility and hardness of the ornament by a multiplication of an adhesion weight, thermal conductivity, and thermal expansion coefficient of the ornament to obtain the ornament physical property score; and
performing minimum-maximum normalization based on a predetermined minimum ornament physical property score and a predetermined maximum ornament physical property score to obtain a normalized ornament physical property score.

19. The method of claim 10, further comprising transmitting the modified three-dimensional dental laminate modeling file to a 3D printer connected to the platform over a wired/wireless network such that the 3D printer fabricates a modified three-dimensional dental laminate.

20. A computer-readable recording medium storing therein a computer program, wherein when the computer program is executed by a server including at least one processor, the computer program causes the at least one processor to perform the method of claim 10.
